(19) United States
(12) Patent Application Publication
(10) Pub. No.: US 2020/0230289 A1
(43) Pub. Date: Jul. 23, 2020

(54) TIME-DELAYED CROSS-LINKING OF TISSUE FILLERS AND APPLICATIONS THEREOF

(71) Applicant: LifeCell Corporation, Madison, NJ (US)

(72) Inventors: Israel James Jessop, Garden Ridge, TX (US); Ming F. Pomerleau, Califon, NJ (US)

(21) Appl. No.: 16/745,633

(22) Filed: Jan. 17, 2020

Related U.S. Application Data
(60) Provisional application No. 62/794,092, filed on Jan. 18, 2019.

(51) Int. Cl. A61L 27/20; A61K 47/42; A61K 47/36; A61L 27/36

(52) U.S. Cl. CPC A61L 27/20; A61K 47/42; A61K 47/36; A61L 2300/11; A61L 2300/236; A61L 2400/06; A61L 2300/254; A61L 27/3604

(57) ABSTRACT

Provided are compositions and methods for achieving time-delayed cross-linking of hyaluronic acid (HA) to be used as a soft tissue filler. The compositions may include at least one component that facilitates cross-linking of HA, which is encapsulated and released in a time-delayed manner. Also provided are methods for treating or augmenting tissue using such compositions.

This application claims priority under 35 USC § 119 to U.S. Provisional Application No. 62/794,092, which was filed on Jan. 18, 2019 and is herein incorporated by reference in its entirety.

The present disclosure relates to tissue fillers, including tissue fillers that can be cross-linked or otherwise modified in a time-delayed manner after implantation.

Soft tissue fillers are used to treat, regenerate, and/or augment tissues. Hyaluronic acid (HA) is a biocompatible filler material that can adapt to various geometries of soft tissue voids and defects. Its flexibility and low immunogenicity when injected into human or animal tissues make it one of the most desirable dermal tissue fillers for medical and cosmetic uses.

Some HA compositions, however, may migrate or flow after implantation. Accordingly, to prevent this problem, HA may be cross-linked soon after injection, thereby causing solidification and preventing migration. Rapid cross-linking, however, may leave very limited time for the fillers to be delivered to the tissues before hardening. Furthermore, the injected filler often cannot be adjusted to ideal shapes and sizes due to the lack of time for molding before complete cross-linking.

Accordingly, there is a need for filler materials or other injectable materials that allow controlled cross-linking or delayed cross-linking of HA so that the material can be better injected and adjusted before it solidifies or becomes too viscous.

The present disclosure relates generally to compositions and methods that can achieve time-delayed cross-linking of hydrogels, e.g., hyaluronic acid, that can be used to treat, regenerate, and/or augment tissue. The present disclosure also relates to methods of treating and/or augmenting tissues using such compositions.

Further features and advantages of certain embodiments will become fully apparent in the following description of embodiments, and from the claims.

### DETAILED DESCRIPTION

Disclosed herein are compositions and methods for achieving time-delayed cross-linking of hyaluronic acid (HA) to be used as a soft tissue filler. In various embodiments, at least one component that facilitates cross-linking of HA is encapsulated in a group of particles and is released in a time-delayed manner to provide a controlled rate of cross-linking. Specifically, disclosed herein is a composition that comprises an HA component and a group of particles encapsulating at least one component that facilitates cross-linking of the HA. Upon contact with water or an aqueous solution, the particles will release the at least one component that facilitates cross-linking.

The HA component and the group of particles can be packaged in separate containers prior to use. The components that facilitate cross-linking of HA, for example, a peroxide and a peroxidase, are not in contact with one another prior to use. They are either encapsulated in different particles, or packaged separately with one component encapsulated in the particles and the other pre-mixed with the HA. Upon mixing with the HA or an aqueous solution (including aqueous body fluids), the particles will release the encapsulated component(s) in a time-delayed manner, and the mixed components (e.g., the peroxide and peroxidase) will facilitate the cross-linking reaction of HA.

The prolonged cross-linking time provides sufficient time for the material to be injected in human or animal tissues and allows in situ adjustment of the injected material.

The materials can be used in the treatment and/or augmentation of hard or soft tissue. The materials can act as biological scaffolds that conform to irregular and/or three-dimensional geometries in vivo, stay in the desired location, retain their volume and structural integrity over time, integrate well with surrounding tissue, and/or promote cell in-growth and regeneration.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, “hyaluronic acid” (HA) is an anionic, nonsulfated glycosaminoglycan. Hyaluronic acid is a polymer of disaccharides composed of D-glucuronic acid (glcA) and N-acetyl-D-glucosamine (glcNAc), linked via alternating β-(1→4) and β-(1→3) glycosidic bonds. Hyaluronic acid polymers can be cross-linked when used as a soft tissue filler. In some embodiments, an HA is a flowable HA component. In some embodiments, an HA is a tyramine-substituted HA (tyrHA).

As used herein, an “acellular tissue matrix” refers generally to any tissue matrix that is substantially free of native cells and/or native cellular components. The acellular tissue matrices of the presently disclosed compositions may be derived from any type of tissue. Examples of the tissues that may be used to construct the acellular tissue matrices of the presently disclosed compositions include, but are not limited to, skin, parts of skin (e.g., dermis), adipose, fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue.

As used herein, “cross-linking” refers to a process that links one polymer chain to another chain or portions of the same polymer chain through covalent bonds or ionic bonds and results in the formation of “cross-linked” polymers. A cross-linked polymer can either be a synthetic polymer or a natural polymer. In some embodiments, a cross-linking reaction is a cross-linking of hyaluronic acid polymers.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any ranges described herein will be understood to include the endpoints and all values between the endpoints.

Any section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

### Hyaluronic Acid

Hyaluronic acid (HA) is also known as hyaluronan. HA is common in biologic materials and is concentrated in specialized tissues such as cartilage, vocal cords, vitreous, synovial fluid, umbilical cord, and dermis. In these tissues, its function is manifold, influencing tissue viscosity, shock absorption, wound healing, and space filling. HA has been shown to influence many processes within the extracellular matrix (ECM) in native tissues including matrix assembly, cell proliferation, cell migration, and embryonic/tissue development.

HA is composed of repeating pairs of glucuronic acid (glcA) and N-acetylglucosamine (glcNAc) residues linked by a β-1,3-glycosidic bond, as shown in the following structure:

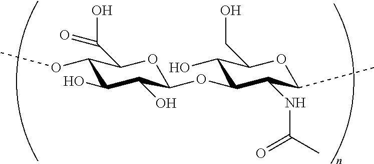

For each HA chain, this simple disaccharide is repeated up to 10,000 times or greater resulting in macromolecules that can have a molecular weight of up to 10 million daltons (i.e., 10 MDa). Therefore, in certain embodiments, the HA component have a molecular weight of up to about 1 kDa, about 2 kDa, about 3 kDa, about 4 kDa, about 5 kDa, about 6 kDa, about 7 kDa, about 8 kDa, about 9 kDa, about 10 kDa, about 20 kDa, about 30 kDa, about 40 kDa, about 50 kDa, about 60 kDa, about 70 kDa, about 80 kDa, about 90 kDa, about 100 kDa, about 200 kDa, about 300 kDa, about 400 kDa, about 500 kDa, about 600 kDa, about 700 kDa, about 800 kDa, about 900 kDa, about 1 MDa, about 2 MDa, about 3 MDa, about 4 MDa, about 5 MDa, about 6 MDa, about 7 MDa, about 8 MDa, about 9 MDa, and about 10 MDa. In certain embodiments, the HA of the presently disclosed compositions have a molecular weight in the range of from about 1 MDa to about 2 MDa. In certain embodiments, the HA of the presently disclosed compositions have a molecular weight in the range of from about 1.5 MDa to about 1.8 MDa.

Adjacent disaccharide units of HA are linked by a β-1,4-glycosidic bond. Each glcA residue has a carboxylic acid group (CO2H) attached to the number 5 carbon atom of the glucose ring. Under biological conditions, HA is a negatively charged, randomly coiled polymer filling a volume more than 1,000 times greater than would be expected based on molecular weight and composition alone. As noted above, the strong negative charges attract cations and water, which allow HA to assume the form of a strongly hydrated gel in vivo, giving it a unique viscoelastic and shock-absorbing property. HA represents a readily available and desirable scaffolding material for tissue engineering applications as it is non-immunogenic, non-toxic, and non-inflammatory. Also, as a naturally occurring extracellular matrix (ECM) molecule, it offers the advantages of being recognized by cell receptors, of interacting with other ECM molecules, and/or of being metabolized by normal physiological pathways.

The presently disclosed flowable hyaluronic acid component can be in any suitable flowable form. In certain embodiments, the flowable hyaluronic acid component can be in any flowable form suitable for injection. In certain embodiments, the flowable hyaluronic acid component is in the form of a liquid, gel, or paste. In certain embodiments, the flowable hyaluronic acid component is in the form of an aqueous liquid, or gel or paste with aqueous solvent. In certain embodiments, the flowable hyaluronic acid component is in the form of a solution, a suspension, a dispersion, or any combination thereof. In certain embodiments, the medium for such solutions, suspensions, and dispersions is water or an aqueous buffer solution. Alternatively, the presently flowable hyaluronic acid component can be in solid form, such as a lyophilized powder, right up until prior to use, when it is then reconstituted to a suitable form for injection (i.e., a solution, suspension, dispersion, or any combination thereof) by addition of water or an aqueous buffer solution to the solid. The presently disclosed compositions can have any viscosity suitable for injection.

In certain embodiments, the HA of the presently disclosed compositions is selected from a group consisting of human-derived HA, porcine-derived HA, bovine-derived HA, bacteria recombinant hyaluronic acid, rooster comb hyaluronic acid or any combination thereof.

In certain embodiments, the HA is a tyramine-substituted hyaluronic acid (tyrHA) derived from the hyaluronic acid (HA) disclosed herein. The glucuronic acid residue provides a carboxyl group periodically along the repeat disaccharide structure of HA that is available for hydroxyphenyl group substitution. The hydroxyphenyl group is introduced by reaction of HA with tyramine.

### Time-Delayed Cross-Linking of HA

Described herein is a composition comprising a flowable hyaluronic acid component and a group of particles encapsulating at least one component that facilitates cross-linking of the hyaluronic acid, wherein the particles are formed from a material that will release the at least one component that facilitates cross-linking of the hyaluronic acid upon contact with water, thereby causing time-delayed cross-linking of the hyaluronic component. Also disclosed herein is a method of delayed cross-linking of a soft tissue filler material, e.g., HA. Other suitable biomaterials can also be used in the composition and methods described herein. Exemplary biomaterials include, but are not limited to, tyramine-conjugated polysaccharides such as chitosan, carboxy methyl cellulose (CMC), chondroitin sulfate, dextrin; and tyramine-conjugated bioresorbable polymers such as poly(lactic acid), poly(lactic-co-glycolic acid), and poly(ethylene glycol).

As disclosed herein, “cross-linking” is a process that links one polymer chain to another or to other parts of the same polymer chain through covalent bonds or ionic bonds and results in the formation of “cross-linked” polymers. Cross-linking of HA can be achieved by chemical cross-linking (e.g., Diels-Alder click cross-linking, and enzymatic cross-linking) or physical cross-linking (e.g., thermos-responsive cross-linking, and ionic cross-linking). In certain embodiments, the cross-linking reaction of HA is facilitated by enzymatic cross-linking. In certain embodiments, the cross-linking reaction of HA is facilitated by an oxidation reaction.

A variety of components can be included to facilitate cross-linking. In certain embodiments, the component that facilitates cross-linking of the HA is a peroxide or superoxide. In certain embodiments, the peroxide is a hydrogen peroxide (H₂O₂). In certain embodiments, the hydrogen peroxide is in a freeze-dried formulation. In certain embodiments, the peroxide is a solid peroxide. In certain embodiments, the solid peroxide is an endoperoxide. In certain embodiments, a solid peroxide is a metal peroxide. Examples of metal peroxides include, but are not limited to, sodium peroxide, zinc peroxide, lithium peroxide, iron peroxide, cadmium peroxide, calcium peroxide, magnesium peroxide and potassium peroxide. Any other peroxides suitable for facilitating cross-linking of HA can be used for the method described herein. In certain embodiments, the peroxide further comprises a stabilizer such as polyvinyl alcohol (PVA) and polyvinyl pyrrolidone (PVP). In certain embodiments, the component that facilitates cross-linking of the HA is a peroxidase. Examples of peroxidases include, but are not limited to, horseradish peroxidase, hematin and soybean peroxidase. In certain embodiments, the cross-linking reaction of HA is facilitated by a peroxide and a peroxidase. Any other components suitable for facilitating cross-linking of HA can be used for the method described herein. In certain embodiments, the one or more components that facilitates the cross-linking of HA are water soluble.

As described herein, the one or more components that facilitates cross-linking of the HA is encapsulated in a group of particles. In certain embodiments, each particle has an outer shell and at least one core materials. In certain embodiments, the at least one core materials are the at least one components that facilitates cross-linking of HA. In certain embodiments, the core material is a stabilized peroxide. In certain embodiments, the core material is a peroxidase. The outer shell of the particles is formed from a material that will release the one or more component upon contact with water or an aqueous solution. In certain embodiments, the outer shells of the particles are water-permeable. In certain embodiments, the particles are dry. In certain embodiments, the particles will release the one or more component upon contact with the flowable hyaluronic acid component. In certain embodiments, the particles will release the one or more component upon contact with an aqueous solution. The aqueous solution can have a pH of about pH 4, about pH 5, about pH 6, about pH 7, about pH 8, about pH 9, or any other pH conditions suitable for injection into human or animal tissues. In certain embodiments, the outer shell of the particles are formed from a material comprising one or more polymer. Examples of the polymers include, but are not limited to, polyvynil alcohol, poly(glycoloic acid), poly(lactic acid), poly(lactic-co-glycolic acid), poly(ethylene glycol), or polycaprylactone. In certain embodiments, the particles are formed from at least one of gelatin, alginate, albumin, collagen, agarose, chitosan, and carrageenan. Any other materials suitable to form the particles can be used for the method described herein. In certain embodiments, the outer shell of all the particles in the composition are formed from the same material. In certain embodiments, the outer shell of the particles in the composition are formed from two or more different materials.

The group of particles can contain one or more particles, but will generally include a number of small particles that can be injected or implanted along with a flowable HA. In certain embodiments, the group of particles are dry. Methods of formulating dry particles include, but are not limited to, spray drying, spray congealing, solvent extraction or double-emulsion evaporation. Other suitable methods of formulating dry particles known in the art can be used in the current invention. The particles can be in any suitable size and shape and in any form suitable for the method described herein. In certain embodiments, the particles are in the form of a dispersion, a suspension, or any combination thereof. In certain embodiments, the particles are dried, polymeric beads. The size of the particles can be, for example, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm in diameter, or any sizes suitable for use in the method described herein.

In certain embodiments, the flowable hyaluronic acid component and the group of particles are packaged in the same container. For example, the hyaluronic acid component is in a dry formulation, e.g. a dry foam and is pre-mixed with the group of particles prior to use. The HA component and the group of particles are not hydrated and the cross-linking of HA is not initiated until immediately prior to injection. In certain embodiments, the flowable hyaluronic acid component and the group of particles are packaged in separate containers prior to use. In certain embodiments, the flowable hyaluronic acid component and the group of particle are not in contact until being mixed in further steps. Examples of containers include, but are not limited to, boxes, tubes, syringes, bags, or any other suitable containers for use in the method described herein. In certain embodiments, the containers are sealed. The containers can be stored at room temperature, or lower temperatures such as 4 ° C., −20 ° C. or −80 ° C., or higher temperatures such as 40 ° C. and 80 ° C.

In certain embodiments, the component that facilitates cross-linking of the HA is a peroxide. The peroxide is encapsulated in the groups of particles as described, and is packaged in a separate container from the hyaluronic acid component. In certain embodiments, the hyaluronic acid component is further mixed with a peroxidase.

In certain embodiments, the component that facilitates cross-linking of the HA is a peroxidase. The peroxidase is encapsulated in the groups of particles as described and is packaged in a separate container from the hyaluronic acid component. In certain embodiments, the hyaluronic acid component is further mixed with a peroxide.

In certain embodiments, the component that facilitates cross-linking of the HA is a peroxide and a peroxidase. The peroxide and the peroxidase are encapsulated in the groups of particles as described and are packaged in a separate container from the hyaluronic acid component. In certain embodiments, the peroxide and the peroxidase are encapsulated in different particles.

In certain embodiments, the composition further comprises a peroxide quencher. The peroxide quencher reacts with the peroxide and quenches residue peroxide after the cross-linking reaction of HA is finished. Therefore the composition being injected in human or animal tissues will not have damaging effects caused by any free peroxide. Examples of peroxide quenchers include, but are not limited to, catalase, chlorine, sulfate, and thiosulfate. In certain embodiments, the peroxide quencher is a catalase. In certain embodiments, the catalase is encapsulated in particles formed from a different material from the particles encapsulating the one or more component that facilitates cross-linking of the HA, therefore the catalase is released at a later time point than the one or more component. In certain embodiments, the catalase is administered after the cross-linking reaction of HA is finished. In an alternative embodiment, the cross-linking of HA is not delayed, and the peroxide quencher is released in a time-delayed manner. In certain embodiments, the peroxide quencher is encapsulated in the group of particles formed from a material that will release the peroxide quencher upon contact with water or an aqueous solution.

As disclosed herein, the composition described herein causes time-delayed cross-linking of the HA. “Time-delayed” or “time-release” is a mechanism that delays the release or delivery of one or more component in a reaction, therefore changes the kinetics and delays the completion of the reaction. In certain embodiments, the reaction time is prolonged in a time-delayed cross-linking of HA. In certain embodiments, a time-delayed cross-linking of HA has a prolonged reaction time before the HA component solidifies. The reaction time of a time-delayed cross-linking of HA can be, for example, about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 60 min, 90 min, 120 min, 180 min, 240 min, or any other duration that is sufficient to complete an injection of the composition in human or animal tissues and an in-situ adjustment of the injected composition. In certain embodiments, the reaction time of a time-delayed cross-linking of HA is at least 30 min before the HA component solidifies. In certain embodiments, the reaction of a time-delayed cross-linking of HA is about 10 to 45 minutes.

n certain embodiments, the at least one component that facilitates the cross-linking of HA is released in a time-delayed manner. In certain embodiments, the peroxide encapsulated in a group of particles is released in a time-delayed manner. In certain embodiments, the peroxidase encapsulated in a group of particles is released in a time-delayed manner. In certain embodiments, the peroxide and peroxidase encapsulated in a group of particles are released in a time-delayed manner. In certain embodiments, the one or more component is released from the group of particles upon contact with water or an aqueous solution.

In certain embodiments, the composition disclosed herein further comprises an acellular tissue matrix. As used herein, the term “acellular tissue matrix” refers generally to any tissue matrix that is substantially free of native cells and/or native cellular components. The acellular tissue matrices of the presently disclosed compositions may be derived from any type of tissue. Examples of the tissues that may be used to construct the acellular tissue matrices of the presently disclosed compositions include, but are not limited to, skin (i.e., dermal), parts of skin (e.g., dermis), adipose, fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue.

The acellular tissue matrices of the presently disclosed compositions can be selected to provide a variety of different biological and/or mechanical properties. For example, an acellular tissue matrix can be selected to allow tissue in-growth and remodeling to assist in regeneration of tissue normally found at the site where the matrix is implanted. In certain embodiments, the acellular tissue matrices of the present disclosure can be selected from ALLODERM® or STRATTICE™ (LIFECELL CORPORATION, Madison, N.J.), which are human and porcine acellular dermal matrices, respectively. In certain other embodiments, the particulate acellular tissue matrix can include CYMETRA®, LifeCell Corporation, Madison, N.J., which is a particulate acellular dermal matrix. In certain other embodiments, the acellular tissue matrix can include demineralized bone matrix (i.e., DBM). Alternatively, other suitable acellular tissue matrices can be used, as described further below.

While an acellular tissue matrix may be made from one or more individuals of the same species as the recipient of the acellular tissue matrix, this need not necessarily be the case. Thus, for example, an acellular tissue matrix may be made from porcine tissue and implanted in a human patient. Species that can serve as recipients of acellular tissue matrix and donors of tissues or organs for the production of the acellular tissue matrix include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice.

In certain embodiments, the acellular tissue matrix can be sterilized prior to use. Sterilization of the acellular tissue matrix can be achieved by any suitable means known in the art. Examples of such means include, but are not limited to, sterilization via e-beam, gamma radiation, UV radiation, and/or supercritical CO₂.

In certain embodiments, the presently disclosed compositions can be formed by thoroughly physically mixing the HA and the acellular tissue matrix components. These components can be mixed by any means known in the art. When they are mixed together, both the HA and the acellular tissue matrix components used to form the presently disclosed compositions can be in any suitable physical form that allows for their mixture with each other and that ultimately does not interfere with the cross-linking of the HA. Examples of such physical forms for the HA include, but are not limited to, solid physical forms, such as a lyophilized powders, and liquid physical forms, such as solutions, suspensions, dispersions, or any combination thereof. In certain embodiments, these solutions, suspensions, or dispersions are aqueous solutions, suspensions, or dispersions. In certain embodiments, the medium for such solutions, suspensions, and dispersions is water or an aqueous buffer solution. Examples of such physical forms for the acellular tissue matrix include, but are not limited to, slurries, diced tissue particles, cryomilled dry powders, micronized dry particles, and freeze dried porous sponge particles. In certain embodiments, the acellular tissue matrix is in the form of a slurry having a solid content in the range of from 10% to 25% by weight and where the liquid medium is an aqueous buffer or a preservation solution.

The HA component can be present in the composition in any suitable concentration. In certain embodiments, the HA component is present in the composition in a concentration of up to 25 mg/mL, based on the total volume of the composition. Examples of such concentrations include, but are not limited to, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, 11.0 mg/mL, 12.0 mg/mL, 13.0 mg/mL, 14.0 mg/mL, 15.0 mg/mL, 16.0 mg/mL, 17.0 mg/mL, 18.0 mg/mL, 19.0 mg/mL, 20.0 mg/mL, 21.0 mg/mL, 22.0 mg/mL, 23.0 mg/mL, 24.0 mg/mL, and 25.0 mg/mL, or ranges therebetween based on the total volume of the composition.

The HA component and the acellular tissue matrix can be present in the composition in any suitable weight ratio to each other. In certain embodiments, the dry weight ratio of HA component and the acellular tissue matrix in the composition is in the range of from 1.0:1.0 to 1.0:100.0. In certain embodiments, the dry weight ratio of HA and the acellular tissue matrix in the composition is in the range of from 1:25 to 1:90. In certain other embodiments, the dry weight ratio of HA and the acellular tissue matrix in the composition is in the range of from 1.0:7.2 to 1.0:36.0. Examples of such concentrations include, but are not limited to, 1.0:7.2, 1.0:8.0, 1.0:9.0, 1.0:10.0, 1.0:11.0, 1.0:12.0, 1.0:13.0, 1.0:14.0, 1.0:15.0, 1.0:16.0, 1.0:17.0, 1.0:18.0, 1.0:19.0, 1.0:20.0, 1.0:21.0, 1.0:22.0, 1.0:23.0, 1.0:24.0, 1.0:25.0, 1.0:26.0, 1.0:27.0, 1.0:28.0, 1.0:29.0, 1.0:30.0, 1.0:31.0, 1.0:32.0, 1.0:33.0, 1.0:34.0, 1.0:35.0, and 1.0:36.0, or ranges found therebetween.

### Methods and Uses

Also disclosed herein is a method of treating or augmenting tissue in a human or an animal comprising selecting a composition comprising a flowable hyaluronic acid component and a group of particles encapsulating at least one component that facilitates cross-linking of the hyaluronic acid, wherein the particles are formed from a material that will release the at least one component that facilitates cross-linking of the hyaluronic upon contact with water, thereby causing time-delayed cross-linking of the hyaluronic component; and mixing the flowable hyaluronic acid component and the group of particles to form a mixture, and introducing the mixture into the tissue of a human or animal to be treated or augmented.

In certain embodiments, the hyaluronic acid component and the group of particles are packaged in the same container. In certain embodiments, the hyaluronic acid component and the group of particles are packaged in separate containers during storage prior to use. In certain embodiments, the particles release the at least one component that facilitates cross-linking of the HA upon contact with water. In certain embodiments, the particles release the at least one component that facilitates cross- linking of the HA upon contact with the flowable hyaluronic acid component. In certain embodiments, the method further comprises providing and mixing an aqueous solution with the hyaluronic acid component and the particles. In certain embodiments, the particle releases the at least one component that facilitates cross-linking of the HA upon contact with the aqueous solution. In certain embodiments, the cross-linking of HA is initiated during mixing.

In certain of those embodiments, the HA component and the particles can be mixed by any suitable means known in the art. Examples of such suitable means include, but are not limited to, syringe-to-syringe luer lock adapter-based systems and in-line static mixers and mixing tips. In certain embodiments, both the HA component and the particles can be simultaneously introduced into the tissue of the human or animal to be treated and/or augmented and mixed to initiate cross-linking. In certain embodiments, the material can be injected into the face using a 27 gauge or smaller needle. In certain embodiments, the material can be delivered to anal fistulae using a cannula.

In certain embodiments, the above method of treating and/or augmenting tissue in a human or an animal involves filling a void in the tissue of a human or an animal. The disclosed method is particularly useful for body contouring procedures. The time-delayed cross-linking of the hydrogels provides sufficient time for the delivery of the material and for molding and manipulation of the injected material to achieve satisfying results. For example, time-delayed cross-linking of the tissue fillers is beneficial for filling large volume voids to resist compression and extrusion during injection. In addition, the described composition and methods are useful for procedures where a more firm filler is desired in order to maintain projection or protrusion where significant skin tension might otherwise flatten or extrude a softer filler. Exemplary suitable uses of the current invention include, but are not limited to, chin augmentation, cheekbone augmentation, thickening of cutaneous tissue cover elbow, knee, or other bony prominence.

In certain embodiments, the void in the tissue is the result of damage or loss of tissue due to various diseases and/or structural damage (e.g., from trauma, surgery, atrophy, and/or long-term wear and degeneration). Examples of such voids include, but are not limited to, simple and complex anal fistulae, osteochondral defects (i.e., defects in bone and/or cartilage), tunneling wounds, hernias (e.g., inguinal hernias), and other deep wounds to both soft (e.g., muscle) and hard (e.g., bone) tissue. Furthermore, the presently disclosed compounds, as well as the resulting cross-linked hydrogels, can also be used to aesthetically (i.e., cosmetically) augment tissue. Thus, in certain other embodiments, the composition can be injected into the tissue of a human and cross-linked to create an aesthetic tissue augmentation implant. Examples of human tissues that can be aesthetically augmented using the presently disclosed compositions include, but are not limited to, breast tissue, buttock tissue, chest tissue, thigh tissue, calf tissue, and facial tissue, including lip, nasolabial folds, and cheek tissue. Examples of particular cosmetic applications for which the presently disclosed compounds, as well as the resulting cross-linked hydrogels, may be used include, but are not limited to, facelift procedures, treatment of facial wrinkles, lines, or other facial features.

1. A composition comprising:
a flowable hyaluronic acid component; and
a group of particles encapsulating at least one component that facilitates cross-linking of the hyaluronic acid;
wherein the particles are formed from a material that will release the at least one component that facilitates cross-linking of the hyaluronic acid upon contact with water, thereby causing time-delayed cross-linking of the hyaluronic acid component.

2. The composition of claim 1, wherein the composition further comprises an acellular tissue matrix.

3. The composition of claim 1, wherein the hyaluronic acid component and the particles are packaged in separate containers.

4. The composition of claim 1, wherein the at least one component that facilitates cross-linking includes a peroxide.

5. The composition of claim 4, wherein the peroxide is a hydrogen peroxide.

6. The composition of claim 4, wherein the peroxide is a solid peroxide.

7. The composition of claim 4, wherein the peroxide is a metal peroxide.

9. The composition of claim 4, further comprising a peroxidase.

10. The composition of claim 9, wherein the peroxidase is contained within the hyaluronic acid component.

11. The composition of claim 1, wherein the at least one component that facilitates cross-linking includes a peroxidase.

12. The composition of claim 12, wherein the peroxidase is a horseradish peroxidase.

13. The composition of claim 9, wherein the peroxide and the peroxidase are encapsulated in different particles.

14. The composition of claim 9, wherein the peroxide is hydrogen peroxide.

15. The composition of claim 9, wherein the peroxide is a solid peroxide.

16. The composition of claim 9, wherein the peroxide is a metal peroxide.

17. The composition of claim 16, wherein the peroxide is selected from the group consisting of zinc peroxide, iron peroxide, or potassium peroxide.

18. The composition of claim 1, wherein the group of particles are formed from a material comprising at least one of polyvinyl alcohol, polyglycoloic acid, polylactic acid, or polycaprylactone.

19. The composition of claim 1, wherein the particles are formed from at least one of gelatin, alginate, albumin, collagen, agarose, chitosan, and carrageenan.

20. The composition of claims 9, wherein the composition further comprises a peroxide quencher.
